(19) United States
(12) Patent Application Publication
(10) Pub. No.: US 2004/0127824 A1
(43) Pub. Date: Jul. 1, 2004

(54) Radiopaque marking pen

(76) Inventor: Mark H. Falahee, Ann Arbor, MI (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/689,124

(22) Filed: Oct. 20, 2003

Related U.S. Application Data
(60) Provisional application No. 60/419,578, filed on Oct. 18, 2002.

(51) Int. Cl. A61F 5/00

(52) U.S. Cl. 602/2

(57) ABSTRACT

A system and method are particularly valuable to performing invasive surgical procedures. In terms of apparatus, a marker in the form of a housing including a reservoir filled with a substance which, when dispensed, is x-ray visible; that is. radiopaque. The substance is preferably also visible to the unaided eye. The substance may be capable of marking on human skin and/or commercially available surgical film. To make the substance radiopaque, a metal or heavy metal such as barium may be used. The substance may be dispensed primarily as a liquid or powder and may be erasable. In all embodiments, the substance is preferably biocompatible and non-toxic. A basic method of preparing for surgical procedure, includes the steps of providing a marker according to the invention, indicating an operative site using the marker, and subjecting the site to x-ray/fluoroscopic exposure for the purpose of comparing the indication to one or more internal structures associated with the procedure.

### REFERENCE TO RELATED APPLICATION

This application claims priority from U.S. Provisional Patent Application Serial No. 60/419,578, filed Oct. 18, 2002, the entire content of which is incorporated herein by reference.

### FIELD OF THE INVENTION

This invention relates generally to surgical procedures and, in particular, to a device for use in making precise markings on a patient for the purpose of making incisions, locating instruments, and so forth.

### BACKGROUND OF THE INVENTION

Minimally invasive surgical techniques are increasingly becoming popular for a variety of surgical procedures. Using these approaches, the surgeon makes relatively small and precise incisions which serve as access points for various surgical instruments, visualization devices, and so forth. Compared to standard or “open” cases, Minimally invasive procedures enable patients to leave the hospital sooner and, on average, experience less pain, and require less medication during and after hospitalization.

According to current practice, marks are made directly on the skin of a patient under x-ray. After the marks are made, a sterile surgical drape is typically placed over the markings and the incisions are made therethrough to perform the operation. This procedure is considered to be somewhat crude, and frequently results in less than the desired accuracy. Additionally, the initial radiation exposure required to make the markings directly on the skin increases patient exposure, which is undesirable.

### SUMMARY OF THE INVENTION

This invention is directed to a system and method particularly valuable to performing invasive surgical procedures. In terms of apparatus, the invention includes a marker in the form of a housing including a reservoir filled with a substance which, when dispensed, is x-ray visible. The substance is preferably also visible to the unaided eye.

The substance may be capable of marking on human skin and/or commercially available surgical film. To make the substance radiopaque, a metal or heavy metal such as barium may be used. The substance may be dispensed primarily as a liquid or powder and may be erasable. In all embodiments, the substance is preferably biocompatible and non-toxic.

A basic method of preparing for surgical procedure, includes the steps of providing a marker according to the invention, indicating an operative site using the marker, and subjecting the site to x-ray/fluoroscopic exposure for the purpose of comparing the indication to one or more internal structures associated with the procedure.

### DETAILED DESCRIPTION OF THE INVENTION

The present invention is directed to a device for use in making precise markings on a patient for making incisions, locating instruments, and so forth. The device and method are particularly useful in performing minimally invasive surgical procedures, though not limited to such uses.

The preferred embodiment resides in a marking pen capable of dispensing radiopaque ink, other fluid, or powders which are also preferably visible to the human eye. Although various formulations may be used the ink composition preferably uses a metal, more particularly a heavy metal such as barium, suspended in a suitable flowable vehicle. To increase human visualization, black or colored pigmented materials may also be added. In all preferred embodiments, the radiopaque material(s), pigments(s) and any other additives are biocompatible and non-toxic. The dispensed “ink” also preferably adheres to a plastic marking surface, such as “Ioband” and other commercially available cut-through surgical films, and may further be erasable.

In use, after covering the operative site with the sterile surgical film, the surgeon can subject the site to x-ray exposure and, by referring to the radiopaque ink, reference markings can be made to indicate deep operative site structures directly onto the plastic surface of the film. This allows the surgeon to make more precise markings to assist in placing surgical instrument access points in the patient which are often necessary in minimally invasive surgical procedures. In situations when a surgeon finds that the first incision marking is not optimal, the erasable embodiment allows the initial marking to be removed and made at another location.

I claim:

1. A marker particularly valuable to performing invasive surgical procedures, comprising:
a housing; and
a reservoir filled with a substance which, when dispensed, is radiopaque.

2. The marking pen of claim 1, wherein the ink is also visible to the human eye.

3. The marker of claim 1, wherein the substance is capable of marking human skin.

4. The marker of claim 1, wherein the substance is capable of marking a commercially available surgical film.

5. The marker of claim 1, wherein the substance includes a metal.

6. The marker of claim 1, wherein the substance includes a heavy metal.

7. The marker of claim 1, wherein the substance includes barium.

8. The marker of claim 1, wherein the substance is primarily a liquid.

9. The marker of claim 1, wherein the substance is primarily a powder.

10. The marking pen of claim 1, wherein the substance is erasable.

11. The marker of claim 1, wherein the substance is biocompatible.

12. The marker of claim 1, wherein the substance is non-toxic.

13. A method of preparing for surgical procedure, comprising the steps of:
providing the marker of FIG. 1;
indicating an operative site using the marker; and
subjecting the site to x-ray exposure for the purpose of comparing the indication to one or more internal structures associated with the procedure.

14. The method of claim 13, wherein the step of indicating an operative site using the marker includes marking a surgical film.

15. The method of claim 13, wherein the step of indicating an operative site using the marker includes marking a surgical film.

16. The method of claim 13, further including the step of erasing the indication if not optimized for the procedure.
